(19) United States
(12) Patent Application Publication
(10) Pub. No.: US 2022/0233358 A1
(43) Pub. Date: Jul. 28, 2022

(54) ELECTROPHYSIOLOGICAL TEST METHOD FOR AUDITORY BRAINSTEM IMPLANT AND RECORDING ELECTRODE USED BY METHOD

(71) Applicant: Shanghai Ninth People's Hospital, Shanghai JiaoTong University School of Medicine, Shanghai (CN)

(72) Inventors: Hao WU, Shanghai (CN); Huan JIA, Shanghai (CN); Ying CHEN, Shanghai (CN)

(73) Assignee: Shanghai Ninth People's Hospital, Shanghai JiaoTong University School of Medicine, Shanghai (CN)

(21) Appl. No.: 17/618,507

(22) PCT Filed: Jun. 12, 2020
(86) PCT No.: PCT/CN2020/095774

§ 371 Date: Dec. 13, 2021

(30) Foreign Application Priority Data
Jun. 13, 2019 (CN) 2019105112487
Jul. 19, 2019 (CN) 2019211396301
Jul. 25, 2019 (CN) 2019106772696
Jul. 26, 2019 (CN) 2019211909232

(51) Int. Cl. A61F 11/04; A61N 1/05; A61N 1/36

(52) U.S. Cl. CPC A61F 11/04; A61N 1/36; A61N 1/05

(57) ABSTRACT

The present invention relates to the field of medical devices, and relates to an electrophysiological test method for an auditory brainstem implant (ABI) and a recording electrode used therein. According to the method of the present invention, there is no need to subcutaneously place an additional recording electrode for a patient, which simplifies preoperative preparation. Moreover, the method has advantages of a high signal-to-noise ratio, a fast response speed, a short recording time, and a large anti-interference ability, thus can effectively improve efficiency of intraoperative electrode test. The method is suitable for use in an auditory brainstem implantation surgery. Besides, the present invention enables the auditory brainstem implantation to be located more accurately, thereby expanding a scope of application.

### TECHNICAL FIELD

The present invention relates to the field of medical devices, specifically to an electrophysiological test method for an auditory brainstem implant (ABI), and a recording electrode used therein.

### BACKGROUND

An ABI is favorable for a patient who is not suitable for a cochlear implantation due to undeveloped cochlea, cochlear ossification, lack of an auditory nerve, and the like. The ABI, which has not been widely used domestically, has a broad application prospect. Good intraoperative monitoring guarantees the effect of postoperative auditory reconstruction.

An ABI device includes two parts: an extracorporeal apparatus and an intracorporeal apparatus. The extracorporeal apparatus includes an electroacoustic transducer, a voice processor, and a connecting wire. The intracorporeal apparatus includes a receiver, an electrode wire, and an electrode array (namely, an auditory brainstem electrode array). A working principle of the ABI is, by placing the electrode array on a surface of a cochlear nucleus in a recess of a fourth ventricle, to directly stimulate a cochlear nucleus complex across a cochlea and an auditory nerve, to produce speech perception and recognition. An ABI implantation surgery is a craniotomy. During the surgery, an implantation area is fully exposed, to well locate the cochlear nucleus. The cochlear nucleus is located in a brainstem and is surrounded by many other nerve nuclei, including a facial nerve nucleus, a trigeminal nerve nucleus, a glossopharyngeal nerve nucleus, etc. Therefore, an accurate implantation of the electrode array is crucial. Any incorrect stimulation to the surrounding structure can result in serious consequences.

At present, electrically-evoked auditory brainstem responses (eABR) is conventionally used for detection after the ABI implantation. The eABR is a far-field potential recording. The electrode array of the ABI emits electrical stimulations. A recording electrode is placed at a top of a head or a mastoid, a reference electrode is placed at two earlobes or a mastoid, a forehead electrode is grounded, and a preamplifier is supposed to be placed close to a subject. A typical response of the eABR occurs within 10 milliseconds after a pulse stimulation, and usually, thousands of average scans are required to obtain a sufficient signal-to-noise ratio. Since the ABI crosses the cochlea and auditory nerve, and accordingly the electrode array directly stimulates the cochlear nucleus, the recording of only partial waves including wave III (cochlear nucleus), wave IV (olive nucleus), and wave V (lateral lemniscus nucleus) can be obtained, which appears 1 to 2 milliseconds (ms) earlier than the recording in a case of using a cochlear implant.

It is important to monitor auditory responses when the electrode array is implanted, which not only indicates a position of the electrode array, but also indicates auditory effect after the implantation. One or more response waves help to confirm that the electrode is implanted correctly, but a process of obtaining eABR is relatively cumbersome. Typically, an external system used for recording is provided and must then be connected/synchronized with a stimulation system. Moreover, various recording electrodes need to be placed on a patient, positions of which may be easily affected by the patient's movement.

### SUMMARY

The present invention provides an automated electrophysiological test method for an ABI, including the following steps: step 1, performing, by a stimulation generator, electrical stimulations on a plurality of ABI electrodes; step 2, sequentially and correspondingly generating, by each of the ABI electrodes, an electrical stimulation signal to stimulate a central auditory system, to generate eABR, and sequentially recording, by a recording electrode in a body of a patient, the generated eABR; and step 3, receiving, by a signal receiving apparatus that is respectively connected to a signal acquisition apparatus and a signal processing apparatus, the eABR recorded by the recording electrode and acquired by the signal acquisition apparatus, and determining, by the signal processing apparatus, whether an eABR target waveform appears at a corresponding ABI electrode through signal superimposition and automatic waveform recognition, to obtain response results of all of the ABI electrodes and display the response results in a three-dimensional image manner.

The present invention further provides an electrophysiological test method for an auditory brainstem implant based on cochlear nucleus action potentials (CNAP), including the following steps: S1, implanting an ABI electrode array; S2, using any one of to-be-tested ABI electrodes on the ABI electrode array as a stimulating electrode to emit an electrical stimulation; S3, using, according to different simulation modes, any other one of the ABI electrodes on the ABI electrode array as a recording electrode of the stimulating electrode, the recording electrode being configured to receive an electrical stimulation signal transmitted by the stimulating electrode and record electrically-evoked cochlear nucleus action potentials; S4, determining whether an electrically-evoked cochlear nucleus action potential target waveform is obtained from a recording result, if the electrically-evoked cochlear nucleus action potential target waveform is obtained in a recorded result, the stimulating electrode being correctly placed, and if the electrically-evoked cochlear nucleus action potential target waveform is not obtained, the stimulating electrode being incorrectly placed, performing fine-tuning on a position of the stimulating electrode, and performing steps S2 to S4 after the fine-tuning, until the target waveform is obtained from the recording result; and S5, determining whether all of the to-be-tested ABI electrodes on the ABI electrode array have been tested: if all of the to-be-tested ABI electrodes on the ABI electrode array have been tested, ending an electrophysiological test process; and if not all of the to-be-tested ABI electrodes on the ABI electrode array have been tested, performing step S2, and testing a next one of the to-be-tested ABI electrodes until all of the to-be-tested ABI electrodes have been tested.

The present invention further provides a non-invasive nerve clamp recording electrode, including: a misaligned and complementary clip, including two clip pieces, front ends of the two clip pieces being misalignedly opened to form an opening at a head of the clip, or the two clip pieces being complementarily closed to form a complete closed loop structure; a plurality of electrodes exposedly arranged at an inner side of the closed loop structure, electrically connected to an external signal generator and/or a signal receiver through a wire; two pressing sections, respectively extending outward from a tail of the clip, and providing a first force for making the clip open by transmitting an external pressing force applied to the two pressing sections; a first elastic body, arranged at rear ends of the clip pieces that are at the tail of the clip and at the pressing sections, an elastic force of the first elastic body being used as a second force for making the clip close; and a second elastic body, arranged at the tail of the clip, two ends of the second elastic body respectively abutting against the two clip pieces, and an elastic force of the second elastic body being used as a third force for making the clip open.

The present invention further provides a cochlear nucleus recording electrode, including: an electrode array, including a body and a plurality of first test electrodes distributed on the same surface of the body; a wire, passing through the body, being connected to the first test electrodes correspondingly, and extending outside the body from a tail of the electrode array to receive an electrical stimulation signal; and a first clampable member, arranged on the wire extending from the tail of the electrode array. Optionally, the cochlear nucleus recording electrode further includes one or more movable electrodes. Each of the movable electrodes is provided with a lead to transmit an electrical stimulation signal, an end of the lead is connected to a second test electrode, and the other end of the lead is arranged at the wire extending from the tail of the electrode array. The lead of each of the movable electrodes is provided with a second clampable member.

### BRIEF DESCRIPTION OF THE DRAWINGS

FIG. 1 is a schematic diagram of an electrophysiological test method for an auditory brainstem implant in a related art.

FIG. 2 is a schematic diagram of an automated electrophysiological test method for an auditory brainstem implant consistent with the present invention.

FIG. 2a is a schematic diagram of waveforms in a case that an ABI electrode has a good response consistent with the present invention.

FIG. 2b is a schematic diagram of waveforms in a case that an ABI electrode has a normal response consistent with the present invention.

FIG. 2c is a schematic diagram of waveforms in a case that an ABI electrode has a poor response consistent with the present invention.

FIG. 3 is a schematic diagram of a relationship between an electrode array and a cochlear nucleus consistent with the present invention.

FIG. 4 is a flowchart of an electrophysiological test method for an auditory brainstem implant based on a CNAP consistent with the present invention.

FIG. 5 is a schematic diagram of a principle of an ABI electrode array performing an electrical stimulation and recording consistent with the present invention.

FIG. 6 is a schematic diagram of a recording result of positive and negative waves caused by the present invention.

FIG. 7 is a top view of a non-invasive nerve clamp recording electrode in a case that a clip is closed consistent with the present invention.

FIG. 8 is a top view of a non-invasive nerve clamp recording electrode in a case that a clip is open consistent with the present invention.

FIG. 9 is a side view of clip pieces in a non-invasive nerve clamp recording electrode being complementarily closed consistent with the present invention (other parts of a clip are omitted).

FIG. 10 is a side view of clip pieces in a non-invasive nerve clamp recording electrode being misalignedly open consistent with the present invention (other parts of a clip are omitted).

FIG. 11 is a schematic diagram of a first elastic body in a non-invasive nerve clamp recording electrode being a torsion spring consistent with the present invention.

FIG. 12 is a schematic diagram of a second elastic body in a non-invasive nerve clamp recording electrode being a coil spring consistent with the present invention.

FIG. 13 is a schematic diagram of a second elastic body in a non-invasive nerve clamp recording electrode being a serpentine spring consistent with the present invention.

FIG. 14 is a schematic diagram of a cochlear nucleus recording electrode provided with a clampable member consistent with the present invention.

FIG. 15 is a schematic diagram of an electrode array in a cochlear nucleus recording electrode having different colors to assist in distinguishing an electrode orientation consistent with the present invention.

FIG. 16 is a schematic diagram of a cochlear nucleus recording electrode provided with a movable electrode consistent with the present invention.

FIG. 17 is an exemplary structural schematic diagram of a clampable member in a cochlear nucleus recording electrode consistent with the present invention.

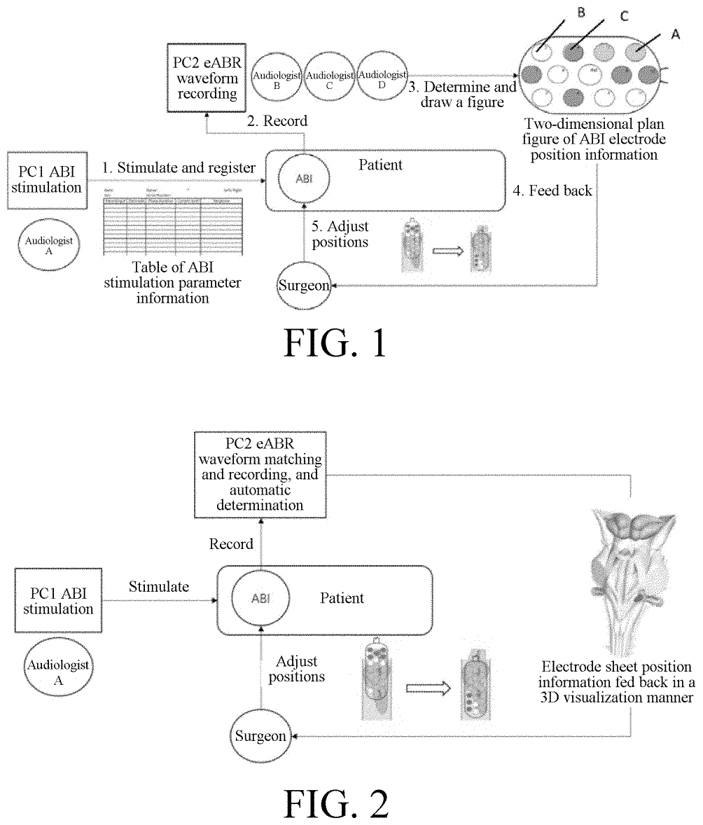

### DETAILED DESCRIPTION OF THE PREFERRED EMBODIMENTS

To make the objectives, technical solutions, and advantages of the embodiments of the present invention more comprehensible, the following clearly and completely describes the technical solutions in the embodiments of the present invention with reference to the accompanying drawings in the embodiments of the present invention.

To make the objectives, technical solutions, and advantages of the embodiments of the present invention clearer, the following clearly and completely describes the technical solutions in the embodiments of the present invention with reference to the accompanying drawings in the embodiments of the present invention. Apparently, the described embodiments are merely some embodiments of the present invention rather than all of the embodiments. All other embodiments obtained by a person of ordinary skill in the art based on the disclosed embodiments without creative efforts shall fall within the protection scope of the present invention.

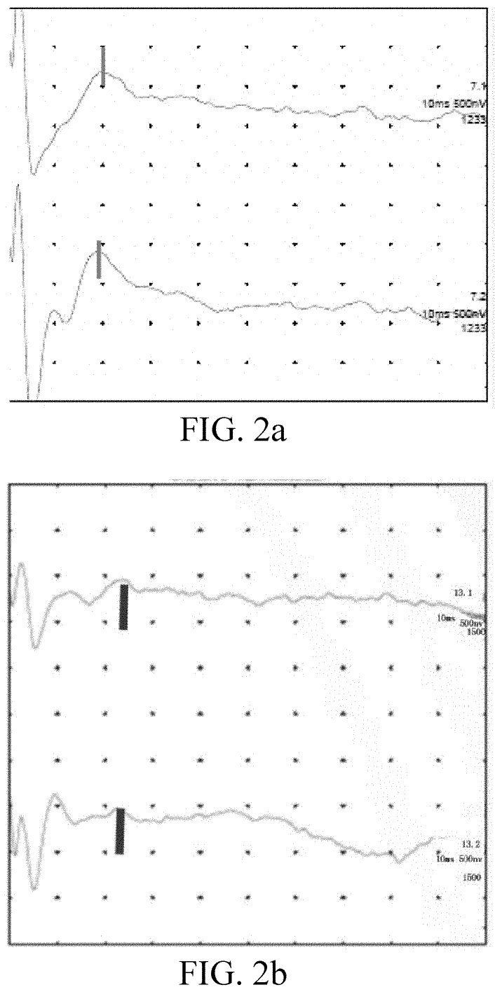

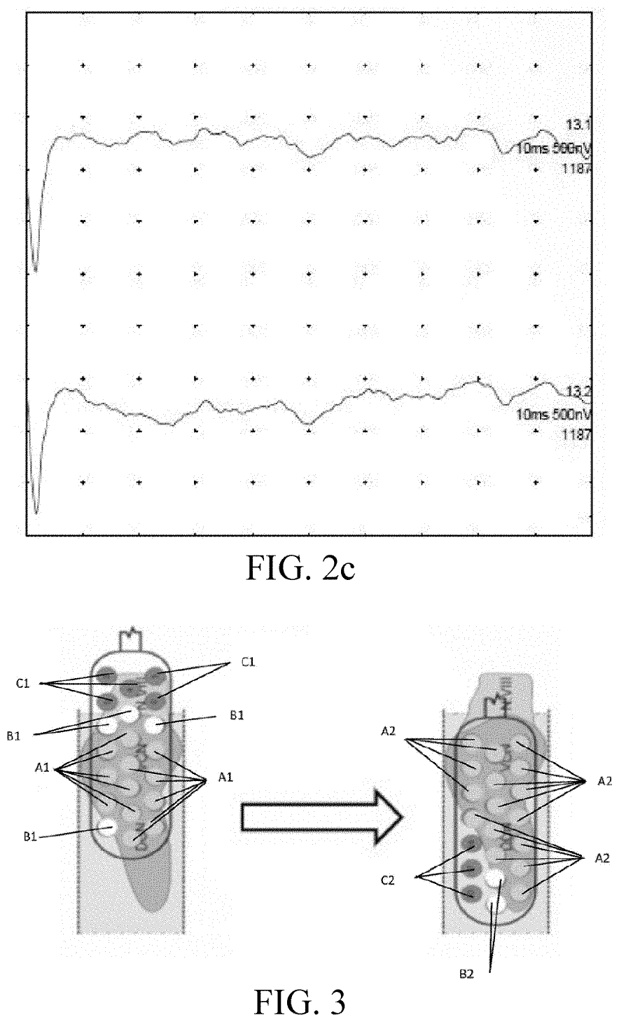

The present invention provides an automated electrophysiological test method for an ABI. As shown in FIG. 2, the method includes the following operations:

S1. Before a surgery, first an electrode group for detecting eABR is placed at a patient's head by an audiologist, the electrode group including a reference electrode placed at a top of the head (a preferred position), a ground electrode placed on a chest skin (a preferred position), and one or more recording electrodes placed in front of both ears (preferred positions). The recording electrode is not limited to being placed at the top of the patient's head, but may also be placed at other parts of the head or at a forehead. Besides, positions of the recording electrodes and the reference electrode may be changed according to an implanter's condition.

S2. During the surgery, a surgery area is exposed by a surgeon, and an eABR detecting is started after auditory brainstem electrodes (ABI electrodes) have been implanted.

Step S2 further includes the following operations:

S21. First, a stimulation generator performs an electrical stimulation on each connected ABI electrode.

In step S21, a first computer 1 (PC1) is electrically connected to the stimulation generator, to control the stimulation generator. The stimulation generator receives a stimulation control signal from the PC1 and transmits an electrical stimulation signal to an ABI electrode.

Generally, there are 12 to 22 ABI electrodes that have been implanted. Each electrical stimulation is only used to stimulate one ABI electrode. An electrical stimulation process of each ABI electrode is performed sequentially until the electrical stimulation processes of all ABI electrodes have been completed. Besides, in an embodiment, a quantity of to-be-tested ABI electrodes is determined by an expert system (for example, a surgeon).

S22. Each ABI electrode correspondingly receives an electrical stimulation signal, and stimulates a central auditory system to generate local potential, so as to obtain eABR.

In step S22, the eABR is one kind of an auditory evoked potential. The eABR can be recorded by the recording electrode placed on the patient. That is, the to-be-tested ABI electrodes are tested sequentially, and the same recording electrode is responsible for all the recording.

S23. Since the eABR have a low signal-to-noise ratio, a signal receiving apparatus is connected to a signal acquisition apparatus, to receive the eABR generated by the central auditory system in the patient's head, which are recorded by the recording electrode and acquired by the signal acquisition apparatus. The signal receiving apparatus is connected to the second computer 2 (PC2, a computer used to match and record eABR waveforms). The PC2 performs filtering, superposition, and other processing (for example, 100 to 1000 times) on the eABR, to form a relatively stable and characteristic target eABR waveform. The “stable” refers to that the eABR waveform after the superimposition has a stable baseline, and basically maintains a consistent form, latency, and amplitude. The “characteristic” refers to that the eABR wave after the superimposition always exists, with a wave crest becoming larger as a stimulus amount is increased and the wave crest becoming smaller as the stimulus amount is decreased. The signal acquisition apparatus is connected to the recording electrode.

In step S23, the eABR waveform is automatically recognized by a software recognition algorithm module in the second computer 2. A starting point of the eABR waveform generally appears within 1 ms, and an entire eABR waveform time limit is approximately within 3 ms, so the software recognition algorithm module can automatically recognize the waveform within the eABR waveform time limit. The software recognition algorithm module also performs a differential calculation to calculate a slope of data points on the eABR waveform, so as to find a starting point, a wave crest, and a wave trough of the waveform, thereby locating and recognizing the entire eABR waveform, and further automatically calculating a latency, amplitude, time limit of the eABR waveform.

S23. In a case that the PC 1 controls the stimulation generator to apply a minimum amount of an electrical stimulation to a certain ABI electrode, if the PC 2 recognizes the stable and characteristic target eABR waveform, the ABI electrode is determined to have a good response; if the PC 2 fails to recognize the eABR waveform, the PC 1 automatically increases the amount of the electrical stimulation, steps S21 to S23 are repeated until the stable and characteristic eABR waveform appears, and then the ABI electrode is considered to have a normal response; and if the amount of the electrical stimulation reaches a maximum amount, but there is still no target eABR waveform that can be recognized by the PC 2, the ABI electrode is considered to have no response.

FIG. 2a shows waveforms in a case that an ABI electrode has a good response, from which it can be seen that the same stimulation intensity always causes waveforms with similar crest values in the same latency, an abscissa indicating a time and an ordinate indicating an amplitude. FIG. 2b shows waveforms in a case that an ABI electrode has a normal response, from which it can be seen that the same stimulation intensity causes similar waveforms in the same latency, but with different crest values. FIG. 2c shows waveforms in a case that an ABI electrode has a poor response, from which it can be seen that there is no relatively stable and characteristic target waveform.

In step S23, the amount of the electrical stimulation (such as the minimum amount of the electrical stimulation, the amount of an electrical stimulation increased each time, and the maximum amount of the electrical stimulation) is determined by an expert system (such as an audiologist).

S24. Perform electrical stimulations on all required ABI electrodes sequentially according to the above steps S21 to S23, and perform automatic recognition and determination. ABI electrodes with good responses or normal responses or no response are obtained by the PC 2 in an automatic determination manner.

S25. The PC 2 may also automatically simulate and draw a figure of positions of the ABI electrodes (electrode array position information in a 3D visualization structure) based on information about the obtained eABR and the eABR waveform, and display the figure on an interface of the PC 2, to facilitate subsequent use in a process of adjusting the positions of the ABI electrodes by a surgeon.

S26. A surgeon may also perform an adjustment on the position of the ABI electrode with a normal response or no response according to result information imaged by the PC 2 (the electrode array position information in the 3D visualization structure), and after the adjustment, the above steps S21 to S23 are repeated until a most suitable position of the ABI electrode has been found, to obtain a good position of the entire electrode array.

In an embodiment, the good position of the entire electrode array is determined by an expert system (for example, a surgeon).

Besides, the PC 1 connected to the stimulation generator, and the PC 2 used to match and record eABR waveforms in the present invention may be implemented by one computer, that is, the stimulation generator and the signal receiving apparatus are both connected to the computer.

FIG. 3 shows a schematic diagram of a relationship between an electrode array and a cochlear nucleus. In the electrode array on the left half of the figure, twelve electrodes (indicated by A1) have good responses and good positions, four electrodes (indicated by B1) have normal responses and normal positions, and five electrodes (indicated by C1) have no response and poor positions. The electrode array after position adjustment becomes as shown in the right half of the figure, in which sixteen electrodes (indicated by A2) have good responses and good positions, two electrodes (B2) have normal responses and normal positions, and three electrodes (C2) have no response and poor positions.

In addition, the forgoing automated electrophysiological test method consistent with the present invention is also applicable to a cochlear implant, which is not detailed herein.

The automated electrophysiological test method for an ABI consistent with the present invention uses the eABR waveform automatic determination manner, and automatically records relevant stimulation information and matched eABR waveforms, automatically simulates and draws an electrode position figure (electrode array position information in a 3D visualization structure), which can replace the conventional manual recording approach. The present test method can effectively improve the efficiency of an audiologist performing electrode testing during a surgery, thereby saving labor. In addition, according to the displayed electrode array position information in the 3D visualization structure, the efficiency of a surgeon adjusting the electrode array position can be improved, which shortens surgical time, reduces surgical risk, and improves patient prognosis. Good intraoperative detection guarantees the effect of postoperative auditory reconstruction; thus the method of the present invention has a broad application prospect.

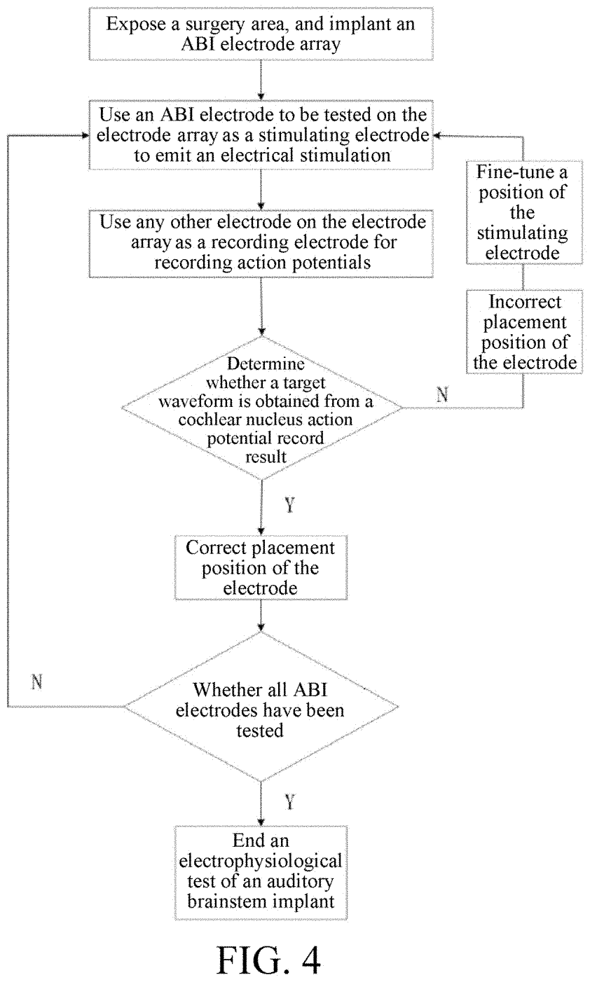

The present invention further provides an electrophysiological test method for an ABI based on electrically-evoked cochlear nucleus action potentials (CNAP). As shown in FIG. 4, the method includes the following operations:

S1′. During a surgery, exposing, by a surgeon, a surgery area, and implanting an auditory brainstem implant (ABI).

In step S1′, the ABI includes an ABI electrode array (also called an electrode array), a reference electrode, and a ground electrode, used for subsequent detecting of the electrically-evoked cochlear nucleus action potentials. The reference electrode is placed at a top of a head (a preferred position), and the ground electrode is placed on a chest skin (a preferred position). During the surgery, the ABI electrode array is placed on a surface of a cochlear nucleus in a recess of the fourth ventricle according to an anatomy, and subsequently the electrophysiological test method is used to check whether the ABI electrode array is correctly placed.

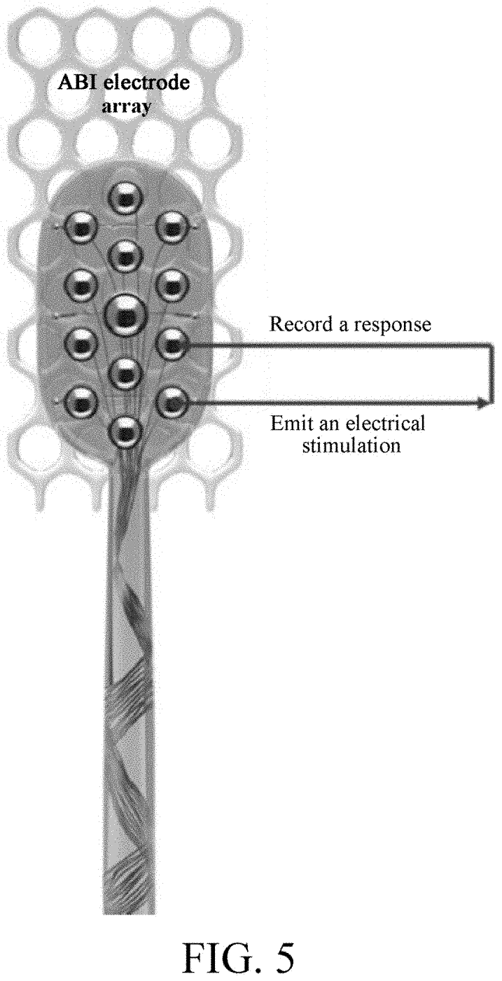

As shown in FIG. 5, the ABI electrode array includes a body and a plurality of to-be-tested ABI electrodes distributed on the same surface of the body.

S2′. Emitting an electrical stimulation by using a certain ABI electrode (a certain to-be-tested ABI electrode) on the ABI electrode array as a stimulating electrode.

S3′. Using any adjacent electrode of the stimulating electrode as a recording electrode, to receive an electrical stimulation signal transmitted by the stimulating electrode, to record cochlear nucleus action potentials.

In step S3′, the recording electrode is connected to a signal acquisition apparatus, for transmitting a cochlear nucleus action potential signal recorded by the recording electrode to a signal processing apparatus.

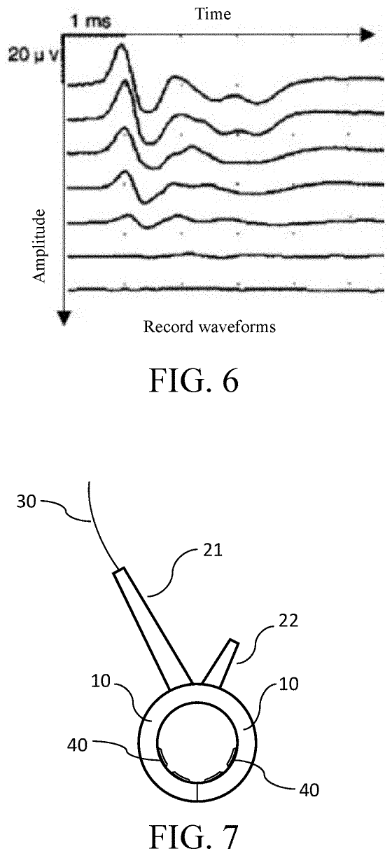

S4′. Determining whether an electrically-evoked cochlear nucleus action potential target waveform is obtained from a recording result in step S3′: if the electrically-evoked cochlear nucleus action potential target waveform is obtained, it indicates that the stimulating electrode is correctly placed; if electrically-evoked cochlear nucleus action potential target waveform is not obtained, it indicates that the stimulating electrode is incorrectly placed, and the position of the stimulating electrode needs to be fine-tuned. The electrophysiology test is performed again after the fine-tuning, that is, steps S2′ to S4′ are repeated until the target positive and negative waveform is generated, which indicates that the stimulating electrode is correctly placed. FIG. 6 shows a schematic diagram of a recording result of positive and negative waves generated by the present invention.

In step S4′, the signal processing apparatus receives the cochlear nucleus action potential signal, and determines whether the electrically-evoked cochlear nucleus action potential target waveform, namely the relatively stable and characteristic electrically-evoked cochlear nucleus action potential waveform, appears at the corresponding stimulating electrode through signal superimposition and automatic waveform recognition. The target waveform refers to a waveform having an obvious crest value within a certain time range, as shown in FIG. 6, an abscissa indicating a time and an ordinate indicating an amplitude. The signal processing apparatus includes a software recognition algorithm module for automatically recognizing the electrically-evoked cochlear nucleus action potential target waveform.

S5′. Determining whether all the to-be-tested ABI electrodes on the ABI electrode array have been tested, if all the to-be-tested ABI electrodes on the ABI electrode array have been tested, ending an electrophysiological test process; and if not, performing step S2′, and continuing a test process of a next ABI electrode until the electrophysiological test process of all the ABI electrodes have been completed.

Generally, there are 12 to 22 electrodes in the implanted ABI electrode array. Referring to the above steps S2′ to S4′, each ABI electrode is used as the stimulating electrode to emit an electrical stimulation, and its adjacent electrode serves as the recording electrode to record action potentials, so as to check whether each ABI electrode is correctly placed, until the electrode stimulation processes of all the ABI electrodes have been completed.

For example, a quantity of the to-be-tested ABI electrodes is determined by an expert system (such as a surgeon).

An electrode that is not adjacent to the stimulating electrode of the present invention may be used as the recording electrode. In a preferred embodiment, the recording electrode is adjacent to the stimulating electrode, which provides a best effect without additional connection to other apparatus. Therefore, different from the conventional eABR test method in which an electrode needs to be subcutaneously placed for a patient, the method of the present invention simplifies preoperative preparation, thereby providing an easier application.

Compared with the related art, the electrophysiological test method of the present invention has the following beneficial effects: (1) the present invention uses the test method in which the electrically-evoked cochlear nucleus action potentials (CNAP) replace the conventional eABR, and has no need to subcutaneously place a recording electrode for a patient, which simplifies preoperative preparation and has advantages of a high signal-to-noise ratio, a fast response speed, a short recording time, and a large anti-interference ability, thereby effectively improving efficiency of intraoperative electrode test; (2) the CNAP consistent with the present invention has advantages as a near-field technology, by using which a signal with a larger amplitude can be observed, and fewer average scans is needed to obtain a satisfactory waveform; and (3) the present invention is also suitable for use in auditory brainstem implantation surgery, which has an easier application.

The electrophysiological test method for an ABI based on CNAP consistent with the present invention has a high signal-to-noise ratio, a fast response speed, a greatly shortened recording time, and a strong anti-interference ability, thus can be used as a standard test method for determining whether an electrode array is correctly placed at a cochlear nucleus. The present invention can also be used to assist post-operative programming of an implantable auditory apparatus. The present invention not only can complete auditory electrophysiological test after an auditory brainstem implant is implanted, but also is more in line with surgical habits, which can shorten surgery time, reduce surgery risk, and improve patient prognosis. The CNAP has advantages as a near-field technology, by using which a signal with a larger amplitude can be observed, and fewer average scans is needed to obtain a satisfactory waveform.

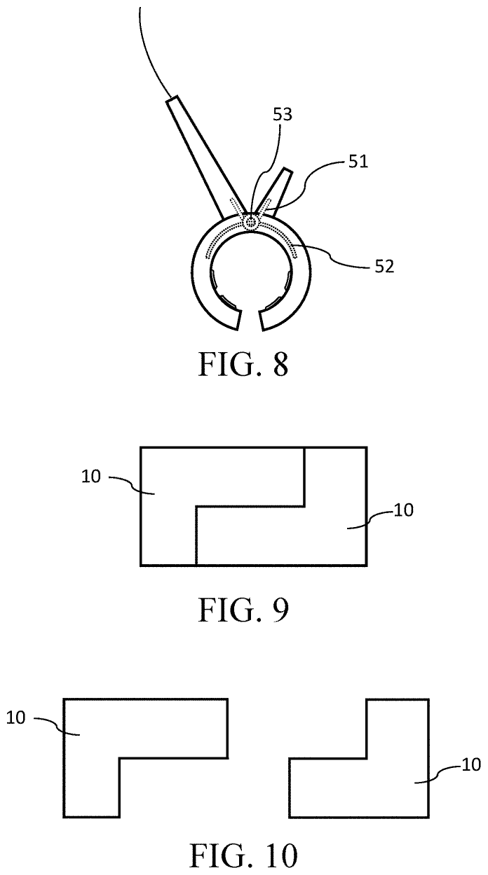

The present invention provides a non-invasive nerve clamp recording electrode. Referring to FIGS. 7 to 10, a body of the non-invasive nerve clamp recording electrode includes a misaligned and complementary clip. That is, two clip pieces 10 are provided, which may be misalignedly opened (FIG. 8 and FIG. 10), or may be complementarily closed to form a complete closed loop structure (FIG. 7 and FIG. 9). An exemplary closed loop structure has a hollow cylindrical shape.

At a head of the clip, in a case that front ends of the two clip pieces 10 are misalignedly opened to a set angle (or beyond the set angle), the clip can clamp a nerve to be monitored. The closed loop structure formed by the two clip pieces 10 embraces the clamped nerve. Unless the two clip pieces 10 are misalignedly opened again to the set angle or beyond the set angle, it is difficult for the nerve to escape from the closed loop structure, which realizes a reliable clamping and fixing.

Several electrodes 40 are exposedly arranged on an inner side of the closed loop structure (FIG. 7), and can be in close contact with the clamped nerve, to transmit an excitation signal to the nerve and/or receive a feedback signal in an electrophysiological monitoring of nerve functions.

The electrodes 40 are electrically connected to an external signal generator and/or a signal receiver through a wire 30. For example, the electrodes 40 may be embedded in or attached to inner sides of the clip pieces 10, so that at least parts of the electrodes 40 are exposed to the inner sides of the clip pieces 10. The wire 30 is firmly connected to the clip pieces 10. For example, the wire 30 may pass through the clip pieces 10, and may also be embedded in or attached to the inner sides or outer sides of the clip pieces 10 (parts where the wire 30 is fixed to the clip pieces 10 and connected to the electrodes 40 are omitted in FIG. 7 and FIG. 8).

The entire closed loop structure may include one or more electrodes 40. In a case that there are a plurality of electrodes 40, the electrodes 40 may be only arranged on one of the clip pieces 10, or arranged on two clip pieces 10, respectively. The electrodes 40 may be symmetrically or asymmetrically distributed. The present invention does not limit the shape and a quantity of the electrodes 40, nor their positions on the clip pieces 10 or the fixing manner.

Rear ends of the two clip pieces 10 are connected or integrated at a tail of the clip. The tail of the clip further extends outward, and is provided with two pressing sections. By relatively pressing the two pressing sections, the front ends of the two clip pieces 10 can be misalignedly opened.

The softness and shape of the entire recording electrode also decide the open/close state of the clip to a certain extent. An O-shaped opening with a slit (FIG. 7) of the clip in the close state becomes a C-shaped (FIG. 8) opening in a case an internal force increasing, and then becomes a U-shape (not shown) in a case of the internal force continuing to increase, which makes the opening to be larger (a larger open angle). For example, a material of the two pressing sections is relatively hard, while a material of the two clip pieces 10 is relatively soft.

Preferably, lengths of the two pressing sections are different. The wire 30 of the electrodes 40 is tightly connected to a first pressing section 21 that is relatively long. For example, the wire 30 may pass through the first pressing section 21 or be embedded in a surface of the first pressing section 21. This can avoid a direct pressing on the wire 30, thereby providing a certain protective effect on the wire 30. A second pressing section 22 is relatively short, which can prevent it from blocking a surgical field of vision during an actual application, thereby not affecting surgical operations.

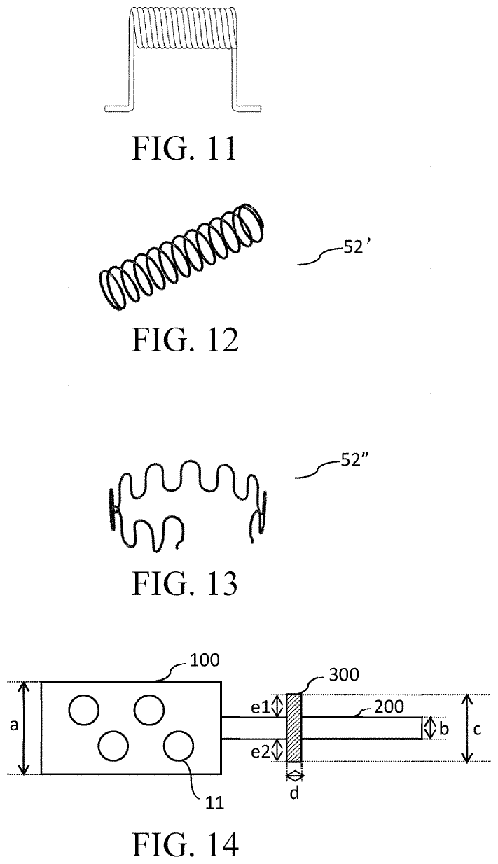

A first elastic body 51 is provided. The first elastic body 51 may be a torsion spring (FIG. 11). A spiral part of the torsion spring is arranged inside the rear ends of the two clip pieces 10, and two torsion arms connecting the spiral part are respectively located in the two pressing sections. An elastic force of the first elastic body 51 makes the clip close.

A second elastic body 52 is provided. The second elastic body 52 may be a coil spring 52′ (FIG. 12), a serpentine spring 52″ (at least one set; FIG. 13), an elastic sheet, or the like, which is arranged in the two clip pieces 10, and fixed on the same axis 53 together with the first elastic body 51. The second elastic body 52 is bent as a whole, with two ends respectively abutting against the two clip pieces 10. An elastic force of the second elastic body 52 is used to make the clip open. The second elastic body 52 may be bent in accordance with a curvature of the clip pieces 10, or the curvature of the second elastic body 52 may be adaptively adjusted according to the elasticity, so that when the clip is in the close state the second elastic body 52 has been deformed to generate a certain elastic force (which is insufficient to open the clip).

Preferably, the first elastic body 51 and the second elastic body 52 are arranged inside the clip (indicated by dashed lines in FIG. 8), so that they are not exposed to the inner sides of the clip pieces 10, to avoid influence on the electrodes 40 in the clip pieces 10. For example, the second elastic body 52 is mainly arranged at the tail of the clip, with no part or only a small part extending to the head of the clip.

The wire 30 of the electrodes 40 is not directly related to the second elastic body 52. Through an adjustment of a design structure and a limited number of tests, the first elastic body 51, the second elastic body 52, and a gravity force of the clip itself may realize:

1) In a case that the pressing sections are pressed to a certain extent, the clip is opened to a set angle that is just for a nerve to enter and exit: in this case, an opening angle of the clip is consistent with a state in which the second elastic body 52 is not deformed, accordingly the elastic force of the second elastic body 52 does not work; at the same time, the first elastic body 51 has not been deformed or an elastic force generated by its deformation is insufficient to actually make the clip close. In other words, there exists a clip opening angle range (namely, the set angle) where the elastic forces of the two elastic bodies do not work, allowing the nerve to enter and exit.

A principle of the above situation is briefly described as follows: before the pressing reaches a certain extent, the clip continues to open as the pressing force increases, and the second elastic body 52 gradually recovers from a deformed state when the clip is closed to a non-deformed state, with its elastic force being gradually reduced. When the clip is opened to the set angle, the clip is out of a space range where the second elastic body 52 works, and the second elastic body 52 is not deformed; in this case, even if the pressing force is removed, the second elastic body 52 does not exert a force on the clip pieces 10. In the above pressing process, the first elastic body 51 has not been deformed or the elastic force generated by its deformation is insufficient to actually close the clip; and if the pressing is removed after the set angle is exceeded, since the first elastic body 51 is sufficiently deformed, its elastic force will actually make the clip close.

The above situation, without considering the influence of the gravity of the clip itself, is applicable to scenarios where the clip is placed horizontally on an object such as a table and is supported by the object; or scenarios where the clip is hold by a user and pressed by the user.

2) Without considering the pressing force, in a case that the clip is in a vertical position, the gravity of the clip itself and the elastic force of the second elastic body 52 work together to make the two clip pieces 10 close (in this case, the first elastic body 51 is not deformed and no force is generated). The vertical position may be defined by an opening direction of the clip that is vertically downward. In this example where the clip is arranged vertically, the two pressing sections are upward (but in other examples, the vertical position of the clip may not be defined by the opening direction, and the pressing sections may be oriented in other directions, which are not limited by the present invention).

3) Without considering the pressing force, in a case that the clip is changed from a vertical position to a position deviated from the vertical position (preferably to a horizontal position), the effect of the gravity is weakened (or the effect of the gravity of the clip itself disappears in the horizontal position), and the second elastic body 52 exhibits an obvious effect (the first elastic body 51 at this time is still not deformed, and no force is generated). In this case, by pulling the wire 30 of the electrodes 40 to drive the clip pieces 10 to move, the clip can be opened to the set angle to release the nerve with the assistance of the second elastic body 52.

According to the non-invasive nerve clamp recording electrode of the present invention, a misaligned and complementary clip structure is formed at the head, to clamp a specific nerve for fixing. Besides, the second elastic body 52 is arranged to provide a guarantee mechanism to avoid clamping too tightly. The second elastic body 52 is cooperated with the first elastic body 51 and the gravity of the clip itself, to enable the clip to maintain a small clamping force as a whole. The clip can be opened to the set angle by pulling the wire 30 of the electrodes 40, so that the nerve can be released without damage. A single electrode 40 or a plurality of electrodes 40 may be provided on the inner side of the clip, to realize various application modes. The present invention is easy to fix, simple to operate, and accurate in recording, which is suitable for neurological monitoring during an intracranial surgery.

The present invention also provides a cochlear nucleus recording electrode for test during an ABI surgery. An auditory brainstem implant apparatus is implanted at a cochlear nucleus, to generate hearing by electrically stimulating the cochlear nucleus. An implantation part of the auditory brainstem implant apparatus includes the cochlear nucleus recording electrode.

As shown in FIG. 14, the cochlear nucleus recording electrode includes an electrode array 100, a wire 200 extending from a tail of the electrode array 100, and a first clampable member 300 arranged on the wire 200. The electrode array 100 includes a body, and a plurality of first test electrodes 11 distributed on the same surface of the body. The wire 200 passes through the body and is connected to the first test electrodes 11 accordingly.

The first clampable member 300 is arranged circumferentially around the wire 200, which is equivalent to that the wire 200 extends radially outward and thereby being thickened. A material of the first clampable member 300 is supposed to be soft enough to not cause any damage to human tissues around an implantation site. Further, a fillet may be provided at a junction between different surfaces of the first clampable member 300 for a smooth transition, so as to avoid sharp parts. Besides, the first clampable member 300 needs to be made of a material with a sufficient strength, to maintain its inherent shape or only allow a small amount of deformation. This is beneficial for a surgical tool to clamp the first clampable member 300, and drive the electrode array 100 at the front of the wire 200 to move to the to-be-monitored cochlear nucleus. The shape, size, and material of the first clampable member 300 may be accordingly adjusted, to satisfy the above requirements as much as possible.

Preferably, the first clampable member 300 has a disc shape, through which the wire 200 passes (FIG. 17). Further, a radial surface and a circumferential surface of the disc may be joined by a fillet to realize a smooth transition. A diameter c of the disc is greater than a diameter b of the wire 200. In different examples, the diameter c of the disc may be less than, equal to, or greater than a width a of the electrode array 100. An axial length d of the disc may be set as required, to facilitate being held by a surgical tool. Or, in some examples, the first clampable member 300 may not be symmetrically arranged with the wire 200 as the center for easy holding and operating during a surgery. For example, a thickness e1 of the first clampable member 300 on one side of the wire 200 may be greater than a thickness e2 of the first clampable member 300 on the other side of the wire 200.

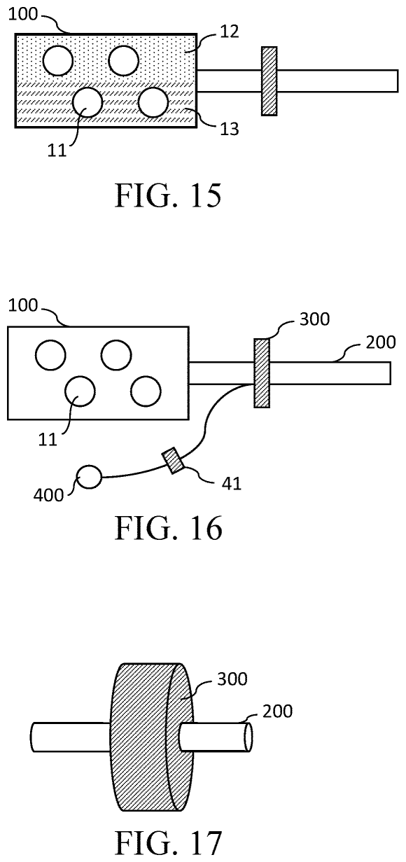

The body of the electrode array 100 on which the plurality of first test electrodes 11 are fixed is usually transparent, so that tissues of human body can be observed through the body during a surgery. A side where the first test electrodes 11 are exposedly arranged is called a front side of the electrode array 100, which usually needs to be attached to a monitored part. In order to quickly distinguish the front and back sides of the electrode array 100 during a surgery, in a preferred example as shown in FIG. 15, an upper half part 12 and a lower half part 13 of the body of the electrode array 100 have different colors (and still have sufficient transparencies). For example, the upper half part 12 of the body is red, and the lower half part 13 is blue. Such color order corresponds to a state where the front side of the electrode array 100 faces forward and the back side of the electrode array 100 faces backward. In this way, the corresponding color order can be observed during a surgery, and if the current order is observed to be the upper half part being blue and the lower half part being red, which is inconsistent with the setting, the body needs to be turned over before being used. Similarly, the left half part and right half part of the body may also have different colors, so as to use an inherent color order (for example, the left half part is red and the right half part is blue) to correspond to the state where the front side of the electrode array 100 faces forward. If the color order is observed to be different, the electrode array 100 needs to be turned over. Therefore, the present invention can use different color for identifications to assist in distinguishing the electrode orientation.

As technologies advance, the electrode array 100 can be made very small to adapt for a small operating space at a cochlear nucleus. The volume of the electrode array 100 can be further reduced by appropriately reducing the quantity of the first test electrodes 11 on the body. For example, one to four first test electrodes 11 are provided on the body of the electrode array 100.

As shown in FIG. 16, in the present invention, one or more movable electrodes 400 may be additionally provided to satisfy various monitoring requirements, serving as a supplement for the first test electrodes 11 on the body. A lead is arranged from the wire 200, such as from a position near the first clampable member 300. An end of the lead is connected to a second test electrode, to form the movable electrode 400. The second test electrode and the first test electrodes 11 on the electrode array 100 may be of the same or different types.

For example, the first clampable member 300 may be provided with a channel through which the lead can pass, so that an initial lead-out angle for the movable electrode 400 is set. A second clampable member 41 may be further provided on the lead of the movable electrode 400, to facilitate intraoperative operations.

The lead of the movable electrode 400 may be one of wires, which merges with other wires 200 extending from the tail of the electrode array 100. Or, the movable electrode 400 may be combined with the electrode array 100 as required. For example, an electrical connector is provided at the first clampable member 300, which is internally connected to one of wires 200, and externally connected to an electrical connector fitted at the other end of the lead, so that the movable electrode 400 can be plugged and unplugged at any time.

The wire 200 extending from the tail of the electrode array 100 may receive the electrical stimulation signal from the stimulation apparatus in a wired or wireless manner, and then transmit the electrical stimulation signal to the first test electrodes 11 on the electrode array 100 (and the second test electrode on the movable electrode 400). The end of the wire 200 is directly connected to the stimulation apparatus; or, the end of the wire 200 is connected to a signal receiving unit, which cooperates with a signal transmitting unit of the stimulation apparatus to receive the electrical stimulation signal.

In accordance with the cochlear nucleus recording electrode provided in the present invention, the overall volume of the electrode array 100 is small; the movable electrode 400 is additionally provided; the body of the electrode array 100 uses different color identifications to assist in distinguishing the electrode orientation; and the first clampable member 300 is provided for easy clamping. The present invention can reduce damage to an implantation site, and is applicable in scenarios such as auditory brainstem implantation surgery and nerve monitoring with simultaneous monitoring of eABR, eCAP and the like, thus having a wide range of applications.

Although the content of the present invention has been described in detail through the above exemplary embodiments, it should be understood that the above description should not be considered as a limitation on the present invention. For a person skilled in the art, various modifications and replacements to the present invention will be apparent after reading the above content. Therefore, the protection scope of the present invention should be subject to the appended claims.

1. An automated electrophysiological test method for an auditory brainstem implant (ABI), comprising:
step 1. performing, by a stimulation generator, electrical stimulations on a plurality of ABI electrodes;
step 2. sequentially and correspondingly generating, by each of the plurality of ABI electrodes, an electrical stimulation signal, to stimulate a central auditory system, to generate electrically-evoked auditory brainstem responses (eABR), and sequentially recording, with recording electrode in body of patient, the generated eABR; and
step 3. receiving, by a signal receiving apparatus that is respectively connected to a signal acquisition apparatus and a signal processing apparatus, the eABR recorded by the recording electrode and acquired by the signal acquisition apparatus, and determining, by the signal processing apparatus, whether an eABR target waveform appears at a corresponding ABI electrode through signal superimposition and automatic waveform recognition, to obtain response results of all of the ABI electrodes and display the response results in a three-dimensional image manner.

2. The automated electrophysiological test method for an ABI as in claim 1, wherein an electrode group for detecting the eABR is placed at a head of the patient, the electrode group comprising a reference electrode placed at a top of the head, a ground electrode placed on a chest skin, and one or more recording electrodes placed in front of two ears.

3. The automated electrophysiological test method for an ABI as in claim 1, wherein the stimulation generator is electrically connected to a control apparatus, and the control apparatus is configured to transmit a stimulation control signal to the stimulation generator, to control the stimulation generator to transmit the electrical stimulation signal to each of the ABI electrodes.

4. The automated electrophysiological test method for an ABI as in claim 1, wherein in step 1, each of the electrical stimulations is only used to stimulate one of the ABI electrodes, and an electrical stimulation process of each of the ABI electrodes is performed sequentially until the electrical stimulation processes of all of the ABI electrodes have been completed.

5. The automated electrophysiological test method as in claim 1, wherein step 3 further comprises:
in a case that a control apparatus controls the stimulation generator to apply a first preset electrical stimulation on a to-be-tested ABI electrode, recording, by the signal processing apparatus, an eABR signal, repeating a step of in a case that a control apparatus controls the stimulation generator to apply a first preset electrical stimulation on a to-be-tested ABI electrode, recording, by the signal processing apparatus, an eABR signal until a preset number of times is reached and ending the step for the signal superposition, performing the waveform recognition, and determining that a response result of the ABI electrode is a first-level expectation result in response to recognizing the eABR target waveform; and/or,
in a case that the control apparatus controls the stimulation generator to apply the first preset electrical stimulation on the to-be-tested ABI electrode, in response to the signal processing apparatus failing to recognize the eABR target waveform, controlling, by the control apparatus, the stimulation generator to automatically increase an amount of the electrical stimulation and repeating steps 1 to 3 until the signal processing apparatus directly recognizes the eABR target waveform, and determining that a response result of the ABI electrode is a second-level expectation result; and/or,
in a case that the control apparatus controls the stimulation generator to apply the first preset electrical stimulation on the to-be-tested ABI electrode, in response to the signal processing apparatus failing to recognize the eABR target waveform, controlling, by the control apparatus, the stimulation generator to automatically increase an amount of the electrical stimulation and repeating steps 1 to 3 until the amount of the electrical stimulation increases to a second preset electrical stimulation, and determining that a response result of the ABI electrode is a third-level expected result in response to the signal processing apparatus still failing to recognize the eABR target waveform.

6. The automated electrophysiological test method for an ABI as in claim 1, wherein in step 3, the signal processing apparatus comprises a software recognition algorithm module, configured to automatically recognize the eABR target waveform, a starting point of the eABR target waveform being within 1 ms and an entire waveform time limit being within 3 ms.

7. The automated electrophysiological test method for an ABI as in claim 6, wherein the software recognition algorithm module is further configured to perform a differential calculation to calculate a slope of data points on the eABR target waveform, to recognize a starting point, a wave crest, and a wave trough of the eABR target waveform, so as to locate and recognize an entirety of the eABR target waveform, and automatically calculate a latency, an amplitude, and a time limit of the eABR target waveform.

8. The automated electrophysiological test method for an ABI as in claim 7, further comprising:
automatically simulating and drawing, by the signal processing apparatus, a three-dimensional image of positions of the ABI electrodes according to acquired information about the eABR and waveforms of the eABR, and displaying the three-dimensional image on an interface of a display module connected to the signal processing apparatus, to be used in an adjustment process of the positions of the ABI electrodes; and
adjusting the position of the ABI electrode whose response result is the second-level expectation result or the third-level expectation result according to the three-dimensional image displayed by the display module, and repeating steps 1 to 3 after the position of the ABI electrode is adjusted, until a position where the response result of the ABI electrode is the first-level expectation result is found, to reach a preset desired result of an entire electrode array position.

9. The automated electrophysiological test method for an ABI as in claim 8, further comprising: adjusting the position of the ABI electrode whose response result is the second-level expectation result or the third-level expectation result according to the three-dimensional image displayed by the display module, and repeating steps 1 to 3 after the position of the ABI electrode is adjusted, until a position where the response result of the ABI electrode is the first-level expectation result is found, to reach a preset desired result of an entire electrode array position.

10. An electrophysiological test method for an auditory brainstem implant (ABI) based on cochlear nucleus action potentials (CNAP), comprising:
S1, implanting an ABI electrode sheet;
S2, using any one of to-be-tested ABI electrodes on the ABI electrode sheet as a stimulating electrode to emit an electrical stimulation;
S3, using any other one of the ABI electrodes on the ABI electrode sheet as a recording electrode of the stimulating electrode, the recording electrode being configured to receive an electrical stimulation signal transmitted by the stimulating electrode and record electrically-evoked cochlear nucleus action potentials;
S4. determining whether an electrically-evoked cochlear nucleus action potential target waveform is obtained: if an electrically-evoked cochlear nucleus action potential target waveform is obtained, the stimulating electrode being correctly placed;
and if an electrically-evoked cochlear nucleus action potential target waveform is not obtained, the stimulating electrode being incorrectly placed, performing fine-tuning on a position of the stimulating electrode, and performing steps S2 to S4 after the fine-tuning, until the electrically-evoked cochlear nucleus action potential target waveform is obtained; and
S5. determining whether all of the to-be-tested ABI electrodes on the ABI electrode sheet have been tested: if all of the to-be-tested ABI electrodes on the ABI electrode sheet have been tested, ending an electrophysiological test process; and if not, performing step S2, and testing a next one of the to-be-tested ABI electrodes until all of the to-be-tested ABI electrodes have been tested.

11. The electrophysiological test method for an ABI based on CNAP as in claim 10, wherein the recording electrode is an adjacent electrode of the stimulating electrode.

12. The electrophysiological test method for an ABI based on CNAP as in claim 10, wherein in step S1, a surgery area is exposed by a doctor during a surgery, and the ABI electrode sheet is placed on a surface of a cochlear nucleus in a lateral recess of a fourth ventricle.

13. The electrophysiological test method for an ABI based on CNAP as in claim 10, wherein the ABI electrode sheet comprises: a body, and the plurality of to-be-tested ABI electrodes that are distributed on the same surface of the body.

14. The electrophysiological test method for an ABI based on CNAP as in claim 10 or 13, wherein a quantity of the to-be-tested ABI electrodes is determined by an expert system.

15. The electrophysiological test method for an ABI based on CNAP as in claim 10, wherein each of the to-be-tested ABI electrodes corresponds to one or more adjacent electrodes, and any of the adjacent electrodes is usable as the recording electrode of the corresponding to-be-tested ABI electrode.

16. The electrophysiological test method for an ABI based on CNAP as in claim 10, further comprising: transmitting, by a signal acquisition apparatus that is connected to the recording electrode corresponding to the stimulating electrode, an electrically-evoked cochlear nucleus action potential signal to a signal processing apparatus, receiving, by the signal processing apparatus, the electrically-evoked cochlear nucleus action potential signal, and determining whether the electrically-evoked cochlear nucleus action potential target waveform appears at the stimulating electrode through signal superimposition and automatic waveform recognition.

17. The electrophysiological test method for an ABI based on CNAP as in claim 16, wherein the signal processing apparatus comprises a software recognition algorithm module, configured to automatically recognize the electrically-evoked cochlear nucleus action potential target waveform.

18. A non-invasive nerve clamp recording electrode, comprising:
a misaligned and complementary clip, comprising two clip pieces, front ends of the two clip pieces being misalignedly opened to form an opening at a head of the clip, or the two clip pieces being complementarily closed to form a complete closed loop structure;
a plurality of electrodes exposedly arranged at an inner side of the closed loop structure, being electrically connected to an external signal generator and/or a signal receiver through a wire;
two pressing sections, respectively extending outward from a tail of the clip, and providing a first force for making the clip open by transmitting an external pressing force applied to the two pressing sections;
a first elastic body, arranged at rear ends of the clip pieces that are at the tail of the clip and at the pressing sections, an elastic force of the first elastic body being used as a second force for making the clip close; and
a second elastic body, arranged at the tail of the clip, two ends of the second elastic body respectively abutting against the two clip pieces, and an elastic force of the second elastic body being used as a third force for making the clip open.

19. The non-invasive nerve clamp recording electrode as in claim 18, wherein the pressing force relatively applied on the two pressing sections makes the clip be in a state where the clip is opened to a set angle, which is consistent with a state where the second elastic body is not deformed, and is also consistent with a state where the first elastic body is not deformed or an elastic force generated by a deformation of the first elastic body is insufficient to make the two clip pieces actually move in a complementary closing direction.

20. The non-invasive nerve clamp recording electrode as in claim 19, wherein in a case that the clip is pressed, a gravity force of the clip is canceled out with a force of an external object carrying the clip, or is canceled out with a force of a user holding the clip; and
the pressing force relatively applied on the two pressing sections makes the clip be in a state where the clip is opened beyond the set angle, which is consistent with a state where the elastic force generated by the deformation of the first elastic body makes the two clip pieces actually move in the complementary closing direction.

21. The non-invasive nerve clamp recording electrode as in claim 18, wherein in a case that the clip is in a vertical position, a gravity force of the clip used as a fourth force for urging the clip to close works together with the elastic force of the second elastic body, to make the clip be in a close state; the close state of the clip is consistent with a state where the first elastic body is not deformed; or,
a state where the clip is deviated from a vertical position is consistent with a state where the first elastic body is not deformed; a gravity force of the clip used as a fifth force for urging the clip to close works together with a first external force applied to the clip and the elastic force of the second elastic body, to make the clip be opened to a set angle; and the fifth force is less than the fourth force; or,
a state where the clip is in a horizontal position is consistent with a state where a gravity force of the clip does not act, and is consistent with a state where the first elastic body is not deformed; a second external force applied to the clip and the elastic force of the second elastic body work together to make the clip be opened to a set angle.

22. The non-invasive nerve clamp recording electrode as in claim 21, wherein the wire of the electrodes is connected to at least one of the pressing sections and at least one of the clip pieces, to further be electrically connected to the electrodes exposed to inner sides of the clip pieces; the first external force comprises a force that pulls the wire of the electrodes to drive the clip to move; the second external force comprises a force that pulls the wire of the electrodes to drive the clip to move; and the second external force is greater than the first external force.

23. The non-invasive nerve clamp recording electrode as in claim 22,
wherein the wire of the electrodes is indirectly connected to the second elastic body.

24. The non-invasive nerve clamp recording electrode as in any one of claims 18 to 23, wherein the front ends of the two clip pieces are misalignedly opened to a set angle, to form the opening at the head of the clip for a nerve to enter and exit; or,
the two clip pieces are complementarily closed to form the complete closed loop structure, to embrace a nerve that enters from the opening, so as to make the electrodes be in close contact with the nerve.

25. The non-invasive nerve clamp recording electrode as in claim 24, wherein in the two pressing sections, a length of the first pressing section is greater than a length of the second pressing section; and the wire of the electrodes is connected to the first pressing section.

26. The non-invasive nerve clamp recording electrode according to claim 24, wherein the second elastic body is coaxially connected with the first elastic body; and the first elastic body and/or the second elastic body are arranged inside the clip, without being exposed to inner sides of the clip pieces.

27. The non-invasive nerve clamp recording electrode as in claim 24, wherein the first elastic body is a torsion spring; the second elastic body is a coil spring, or a serpentine spring, or an elastic sheet; a close state of the clip is consistent with a state where the second elastic body is deformed; and the second elastic body is bent as a whole.

28. A cochlear nucleus recording electrode, comprising:
an electrode sheet, comprising a body, and a plurality of first test electrodes distributed on the same surface of the body;
a wire, passing through the body, being connected to the plurality of first test electrodes correspondingly, and extending outside the body from a tail of the electrode sheet to receive an electrical stimulation signal; and
a first clampable member, arranged on the wire extending from the tail of the electrode sheet.

29. The cochlear nucleus recording electrode as in claim 28, further comprising one or more movable electrodes; wherein each of the movable electrodes is provided with a lead to transmit an electrical stimulation signal, an end of the lead is connected to a second test electrode, the other end of the lead is arranged at the wire extending from the tail of the electrode sheet; and the lead of each of the movable electrodes is provided with a second clampable member.

30. The cochlear nucleus recording electrode as in claim 29, wherein the first clampable member is provided with a channel through which the lead of each of the movable electrodes passes.

31. The cochlear nucleus recording electrode as in claim 28, wherein the body of the electrode sheet comprises a plurality of parts; each of the parts has a different color and a sufficient transparency; a first position order of the plurality of parts corresponds to a first order combination of different colors, which corresponds to a state of a front side of the electrode sheet; a second position order of the plurality of parts corresponds to a second order combination of different colors, which corresponds to a state of a back side of the electrode sheet.

32. The cochlear nucleus recording electrode as in claim 31, wherein the plurality of parts of the body comprise an upper half part and a lower half part with different colors; or, the plurality of parts of the body comprise a left half part and a right half part with different colors.

33. The cochlear nucleus recording electrode as in claim 28, wherein the wire extending from the tail of the electrode sheet is connected to a stimulation apparatus that is configured to provide the electrical stimulation signal;
or, the wire extending from the tail of the electrode sheet is connected to a signal receiving unit; a signal transmission unit of a stimulation apparatus is configured to wirelessly transmit the electrical stimulation signal to the signal receiving unit.

34. The cochlear nucleus recording electrode as in claim 28, wherein the first clampable member is arranged around a circumference of the wire; and the wire is passed through a center of the first clampable member, or passed through the first clampable member from an off-center part.

35. The cochlear nucleus recording electrode as in claim 28, wherein the first clampable member is a disc.

36. The cochlear nucleus recording electrode as in claim 28, wherein there are 1 to 4 first test electrodes on the body of the electrode sheet.
